(19) United States
(12) Patent Application Publication
(10) Pub. No.: US 2007/0014735 A1
(43) Pub. Date: Jan. 18, 2007

(54) Buccal delivery system

(76) Inventor: David D. Mundschenk, Dania, FL (US)

Correspondence Address: LOTT & FRIEDLAND, P.A., ONE EAST BROWARD BLVD., SUITE 1609, FORT LAUDERDALE, FL, 33301, US

(21) Appl. No.: 11/415,377

(22) Filed: May 1, 2006

Related U.S. Application Data
(63) Continuation of application No. 09/240,455, filed on Jan. 29, 1999, now abandoned.

(51) Int. Cl. A61K 39/00; A61L 9/04; A61K 31/14

(52) U.S. Cl. 424/45; 424/184.1; 514/643

(57) ABSTRACT

A delivery formulation and system for delivering inactivated bioactive peptides to the body. The formulation includes effective amounts of the peptide as well as a mucosal permeation enhancer selected from the group consisting of quaternary ammonium salts. The system can be used by spraying the formulation into the buccal cavity, e.g., to the roof of the mouth.

### TECHNICAL FIELD

The present invention relates to methods and systems for delivering medicaments to the body, and in particular, to mucosal membranes and surfaces. In another aspect, the invention relates to medicament and other formulations that include the use of quaternary ammonium salts, such as benzalkonium chloride. In yet another aspect, the invention relates to the delivery of peptides to the body, including inactivated bioactive peptides.

### BACKGROUND OF THE INVENTION

A variety of approaches have been described for use in delivering medicaments to the body, including intravaneously, orally, and topically. Several of such approaches involve nasooral delivery, including delivery to mucosal surfaces by the use of sprays, tablets, devices and the like.

The mouth, also known as the oral or buccal cavity, is placed at the start of the alimentary canal. Anatomically, Gray's Anatomy describes the mouth as consisting of two parts, including an outer, smaller portion, the vestibule (vestibulum oris), and an inner, larger part, the cavity proper (cavum oris proprium). The vestibule is the slit-like aperture bounded in front and laterally by the lips and cheeks, and behind and internally by the gums and teeth. Above and below, the vestibule is limited by the reflection of the mucous membrane from the lips and cheeks, to the gums covering the upper and lower alveolar arch, respectively. The vestibule receives the secretion from the parotid glands and communicates, when the jaws are closed, with the cavity of the mouth by an aperture on each side behind the wisdom teeth.

The cavum oris proprium is bounded laterally and in front by the alveolar arches with their contained teeth, and behind it communicates with the pharynx by a constricted aperture (isthmus faucuim). It is roofed by the hard and soft palate, while the greater part of the floor is formed by the tongue, the remainder being completed by the reflection of the mucous membrane form the sides and under surface of the tongue, to the gum lining the inner aspect of the mandible. The mucous membrane lining the mouth is continuous with the free margin of the lips, and with the mucous lining of the pharynx behind. It is generally of a rose pink tinge and covered by stratified epithelium.

Companies such as Theratech, Inc., for instance, are actively developing systems for the delivery of medicaments via mucosal surfaces. See, for instance, U.S. Pat. No. 5,766,620 for “Buccal delivery of glucagon-like insulinotropic peptides”. This patent describes drug delivery systems and methods for administering a glucagon-like insulinotropic peptide to the buccal mucosa for transmucosal drug delivery are described. The drug delivery systems comprise a drug composition containing an effective amount of the glucagon-like insulinotropic peptide and an effective amount of a permeation enhancer for enhancing permeation of glucagon-like insulinotropic peptide through the buccal mucosa and means for maintaining the drug composition in a drug transferring relationship with with buccal mucosa. These systems can be in free form, such as creams, gels, and ointments, or can comprise a device of determined physical form, such as tablets, patches, and troches. A preferred glucagon-like insulinotropic peptide is GLP-1 (7-36)amide. The '620 patent describes penetration enhancers selected from the group consisting of an organic solvents, cell-envelope disordering compounds, steroidal detergents, bile salts, chelators, surfactants, non-surfactants, fatty acids, and mixtures thereof.

See also, U.S. Pat. No. 5,859,048 (“Pharmaceutics for mucosal administration”), which describes pharmaceutics for mucosal administration containing pharmacologically active peptides or proteins and tolmetin or salts thereof as mucosal absorption enhancers, as well as U.S. Pat. No. 5,827,525 (“Buccal delivery system for therapeutic agents”); U.S. Pat. No. 5,650,192 (“Method for manufacturing buccal delivery device”); U.S. Pat. No. 5,849,322 (“Compositions and methods for buccal delivery of pharmaceutical agents”); U.S. Pat. No. 5,766,620 (“Buccal delivery of glucagon-like insulinotropic peptides”); U.S. Pat. No. 3,948,254 (“Novel drug delivery device”); and U.S. Pat. No. 5,726,154 (“Stabilization and oral delivery of calcitonin”).

On a related subject, quaternary ammonium salts are commonly used for their preservative and other functions. Benzalkonium chloride, for instance, is a quaternary ammonium salt with antiseptic properties and uses similar to other cationic surfactants. According to Sigma product literature, “the mode of action of quaternary ammonium compounds appears to be associated with the agent's effect on the cytoplasmic membrane, which controls cell permeability.” Hence benzalkonium chloride is widely used as a preservative agent in topical, nasal and ocular formulations at concentrations that range from 0.01% to 0.1% for cleansing of wounds and skin.

See also U.S. Pat. No. 5,578,567 (assigned to Sandoz), which describes a nasal pharmaceutical composition, comprising hPTH, in a product commercially available as Miacalcin®. The patent describes the optional use of a variety of additives, including preserving agents such as benzalkonium chloride.

On a separate subject, Applicants have developed a unique class of active agents, termed “immunokines”, which include bioactive peptides, such as toxins, that have been prepared (e.g., by biosynthetic means) or obtained naturally and rendered inactive, e.g., by ozone treatment, to remove some or all of their disulfide linkages. See, for instance, PCT/US97/08074 (International Publication. No. WO 97/43407), the disclosure of which is incorporated herein by reference.

### SUMMARY OF THE INVENTION

The present invention provides a method and system for administering a macromolecular drug to mucosal surfaces of the buccal cavity. The method and system can be used to deliver such drugs to other mucosal surfaces as well, including vaginal, rectal and nasal surfaces. In a preferred embodiment, the method and system include the preparation and delivery of a formulation adapted to contact and adhere to the mucosal tissue of the buccal cavity, and preferably the hard and soft palate of the roof of the mouth. The delivery formulation comprises an immunokine, as defined herein, in combination with an effective amount of a mucosal absorption enhancer selected from the group consisting of quaternary ammonium salts such as benzalkonium chloride.

In a preferred embodiment, the delivery formulation comprises an effective amount of a mucosal absorption enhancer selected from the group consisting of quaternary ammonium salts, and an effective amount of an inactivated bioactive macromolecule (“immunokine”), e.g., having a molecular weight of at least 500 daltons. The ability to deliver immunokines in this manner, and without the need for complex, expensive, potentially unstable mucosal absorption formulations or devices, greatly facilitates the use of such medicaments.

Applicant has discovered that salts such as benzalkonium chloride have a surprising effect in increasing the absorption of immunokines when delivered to the buccal cavity. This is particularly surprising since benzalkonium chloride is not commonly considered a permeation enhancer, yet in turn, the delivery formulation described herein can be used and is efficacious without the need for other, conventional enhancers or devices. Hence the patient can himself quickly and easily deliver an effective amount of the active agent, with little more that a short aerosol spray into the mouth.

The formulation can be delivered (e.g., by spraying, applying, or device (e.g., patch)) to mucosal surfaces such as the roof of the mouth. In a preferred embodiment, the formulation is provided in an aerosol container in order to be sprayed onto a mucosal surface within the buccal cavity, e.g., to the roof of the mouth.

### DETAILED DESCRIPTION

Quaternary ammonium salts useful in the system and method of this invention include those commonly used and considered as safe for human use. Such compounds are typically tetrasubstituted ammonium salts in which the substituent groups are preferably hydrocarbon compounds attached to the nitrogen by an N—C bond, and selected from the group consisting of substituted and unsubstituted, saturated and unsaturated, aliphatic and aromatic, and branched and normal chain groups. In all cases the nitrogen atom is pentavalent and is in the positively charged portion of the molecule, thus quaternary ammonium salts are cationic electrolytes.

A particularly preferred mucosal absorption enhancer of this invention is benzalkonium chloride, also known as alkyl dimethylbenzyl ammonium chloride, alkyldimethyl(phenylmethyl) Quaternary Ammonium Chloride, Ammonyx, and Roccal. BC is commercially available in suitable form from a number of sources, including Sigma Chemical Co. as Product No. B1383.

Quaternary ammonium chlorides are used in an amount effective to increase the permeability and uptake of the immunokine, as compared to a formulation lacking the enhancer. In a preferred embodiment, the QAC is used at a final concentration of between about 0.001% and about 0.1%, by weight, and preferably between about 0.005% and about 0.05%, based on the weight of the formulation.

The system and method of this invention involve the use of a proteinaceous medicament, preferably in the form of an immunokine. The word “immunokine”, as used herein, will refer to an inactivated bioactive polypeptide, i.e., a polypeptide that has had some or all of its native tertiary structure altered by the failure to form one or more disulfide linkages. As used in this sense, immunokines are typically natural molecules (either recovered from natural sources or synthetically produced) and denatured by exposure to ozone or other oxidizing agents. These denatured molecules lack several functions associated with the native parent molecule and have potential applications in the treatment of numerous diseases, particularly neurological diseases (e.g., multiple sclerosis, amyotrophic lateral sclerosis, viral diseases (e.g., herpes, hepatitis) and cancer.

In one embodiment, the immunokine is prepared by a method comprising the steps of:

a) identifying a polypeptide having a biological activity dependent on the presence of one or more disulfide bridges in its tertiary structure,

b) preparing a cDNA strand encoding the polypeptide,

c) expressing the cDNA under conditions in which the polypeptide is recovered in an inactive form due to the failure to form one or more disulfide bridges, and

d) recovering the inactive polypeptide and formulating it into a composition suitable for parenteral administration to a host.

In a further aspect, the invention provides a method of administering a composition comprising an inactivated bioactive polypeptide to the buccal surfaces of a host, comprising the step of providing the polypeptide in an inactive form and in a composition that includes a quaternary ammonium salt, in order to facilitates the administration of the active to the buccal surfaces of a host. In a related aspect, the invention provides a host having administered such a composition.

In another aspect, the invention provides a composition comprising a bioactive polypeptide that has been rendered inactive by virtue of the failure to form one or more of its disulfide bridges. In a related aspect, the invention provides a composition for in vivo administration comprising a bioactive polypeptide that has been inactivated in the manner described herein.

The method can be used to prepare a variety of bioactive polypeptides, including “Group I neurotoxins” (namely, toxins affecting the presynaptic neurojunction), Group II neurotoxins (namely those affecting the postsynaptic neurojunction), and Group III neurotoxins (those affecting ion channels). cDNA sequences for such polypeptides are generally known, or can be determined using conventional techniques.

The cDNA can be expressed using any suitable expression system, under conditions in which the product can be recovered with one or more disulfide bridges unformed. Suitable expression systems include heterologous host systems such as bacteria, yeast or higher eucaryotic cell lines. Examples of useful systems are described, for instance, in “Foreign Gene Expression in Yeast: a Review”, Romanos, et al., Yeast, 8:423-488 (1992). See also, “Yeast Systems for the Commercial Production of Heterologous Proteins”, Buckholz, et al., Bio/Technology 9:1067-1072 (1991), the disclosures of both Romanos et al. and Buckholz et al. being incorporated herein by reference.

These articles are generally directed at the more common goal of affirmatively achieving posttranslational processing and extracellular secretion. Under such conditions, the formation of appropriate disulfide linkages would be included as a necessary step. Given the present description, however, these articles, and the techniques described therein, will be of considerable use to those skilled in the art in achieving the recovery of the unfolded product, e.g., by intracellular expression in yeast.

Preferably, the cDNA is expressed using a yeast expression system, such as Saccharomyces cerevisiae and Pichia pastoris. More preferably, the cDNA is expressed in a Pichia expression system under conditions in which the product is cytoplasmically produced, as opposed to extracellularly secreted. In an exemplary embodiment, the polypeptide is expressed using a Pichia expression system, under conditions in which the leader sequence of naturally-occurring cDNA is removed and replaced with only the initiation codon.

Polypeptides of the present invention are generally stable under suitable conditions of storage and use in which the disulfide bonds are prevented from spontaneously reforming, or are allowed to reform in a manner that precludes the undesirable activity of the polypeptide. Optionally, and preferably, once the inactive polypeptide has been recovered, it is treated by suitable means to ensure that the cysteine residues do not spontaneously reform to form disulfide bridges. An example of a preferred treatment means is the use of ozone treatment as described herein. Alternatively, as will be described in greater detail below, ozone treatment can itself be used to selectively break (i.e., oxidize) the disulfide bonds of a native or recombinantly prepared toxin molecule in order provide a stable, inactive form thereof.

In another optional, and alternative, embodiment a polypeptide such as neurotoxin is produced in an inactive form using the Pichia expression system described herein.

The delivery method and composition of the present invention provide a unique and valuable tool for the synthesis, recovery and delivery of bioactive polypeptides in a manner capable of achieving efficacious dosages, while diminishing undesirable activity, yet retaining other useful properties of the polypeptide (such as immunogenicity and antiviral activity).

As used herein, the following words (and inflections thereof) and terms will have the meanings ascribed to them below:

“bioactive” will refer to a polypeptide capable of eliciting at least one biological response when administered in vivo.

“polypeptide” will refer to any biomolecule that is made up, at least in part, of a chain of amino acid residues linked by peptide bonds.

“inactive” will refer to a polypeptide that is provided in a form in which at least one form of its bioactive responses is substantially terminated or decreased to a desired extent.

“neurotoxin” will refer to a bioactive polypeptide wherein at least one activity (e.g., binding to the acetylcholine receptor) produces a toxic effect on the nervous system of a mammalian host.

The method of the present invention involves an initial step of identifying a bioactive polypeptide having a tertiary structure in which bioactivity is dependent, at least in part, on the formation of one or more disulfide bridges between cysteine residues. Typically, the polypeptide will be one that is naturally secreted in the course of its synthesis, since it is the secretion process that will provide the necessary posttranslational steps, including disulfide bond formation. Preferably, the polypeptide is one that is stable when recovered and that retains other desirable properties in the unfolded state, such as immunogenicity and/or antiviral, anti-tumor or wound healing activity.

The amino acid sequence and tertiary structure of a number of bioactive polypeptides is known. Suitable polypeptides include those in which one or more disulfide bridges are known to form in the natural configuration, and in which such bridge(s) are necessary for the bioactivity of the polypeptide. Such bridges can be of either an intramolecular (i.e., within a single polypeptide) nature and/or an intermolecular (e.g., between discrete subunits) nature.

Secreted or cell-surface proteins often form additional covalent intrachain bonds. For example, the formation of disulfide bonds between the two —SH groups of neighboring cysteine residues in a folded polypeptide chain often serves to stabilize the three-dimensional structure of the extracellular proteins. Protein hormones such as oxytocin, arginine vasopressin, insulin, growth hormone and calcitonin, all contain disulfide bonds. Enzymes such as ribonuclease, lysozyme, chymotrypsin, trypsin, elastase and papain also have their tertiary structure stabilized by disulfide bonds. Besides the bioactive proteins listed above, there are numerous other proteins that contain disulfide bonds, such as the immunoglobulins (IgA, IgD, IgE, IgM), fibronectin, MHC (major histocompatible complex) molecules and procollagen. Many polypetides from animal venoms also contain disulfide bonds.

In a preferred embodiment, the method of the present invention is used to prepare inactivated forms of neurotoxins, and more preferably neurotoxins from amongst the four groups provided below. As described above, those in Group I typically affect the presynaptic neurojunction, those in Group II typically affect the postsynaptic neurojunction, and those in Group III typically affect ion channels. Lastly, there are also included toxins known only to have a toxic affect by causing membrane damage.

| Neurotoxins | | | Membrane- |
|---|---|---|---|
| Group I | Group II | Group III | damaging toxins |
| notexin | α-conotoxin | dendrotoxins | myotoxins |
| β-bungarotoxin | α-cobrotoxin | scorpion toxins | cardiotoxins |
| crotoxin | erabutoxin | μ-conotoxins | mellitin |
| taipoxin | α-cobratoxin | sea anemone toxins | phospholipases |
| textilotoxin | α-bungarotoxin | omega conotoxins | |
| α-latrotoxin | | apamin | |
| | | (from bee venom) | |
| | | mast cell | |
| | | degranulating | |
| | | peptide | |

The method involves a further step of preparing or isolating a corresponding gene (e.g., a cDNA strand) encoding the polypeptide. Using the primary amino acid sequence discussed above, and in view of the present teaching, those skilled in the art will appreciate the manner in which such polypeptides can be synthesized using genetic engineering techniques. Generally, and preferably, one or more of the native control (e.g., leader) sequences of the desired cDNA are removed and replaced with one or more corresponding sequences in order to facilitate the desired expression.

Polypeptide components from animal venoms, for instance, can be obtained from the animals themselves or from other sources, or they can be created in the laboratory using conventional protein engineering techniques. In the former approach, animals are induced by mechanical or electrical stimuli to release venom from their glands, which travels through a venom canal and out the fang or stinger. The venom is collected and various constituents of the venom are purified by conventional chromatographic techniques.

In the latter approach, constituents from the venom are synthesized by cloning the genes encoding the various polypeptide elements and expressing these genes in heterologous host systems such as bacteria, yeast or higher eucaryotic cell lines. Yeast expression systems are presently preferred, since they tend to provide an optimal combination of such properties as yield and adaptability to human use products.

Expressed products are then purified from any other contaminating host polypeptides by means of chromatographic techniques similar to those used to isolate the polypeptides directly from the venom.

There are significant advantages to the use of host systems other than the venomous animals to obtain the venom components. The danger to human lives in obtaining the venom from the animal is eliminated. There will no longer be a need for the costly animal husbandry required to maintain venomous animals for venom extraction. The quantities of materials that can be obtained from the genetic engineering approach can be one or more orders of magnitude greater than the quantities that can be derived from the venom itself. Moreover, once the gene(s) is cloned and expressed, it can be used to provide a continual, reproducible source in the form of a bacterial, yeast or higher eucaryotic cell line seed culture.

Seed cultures can be stored and transported in the frozen state, lyophilized, or, in some cases, plated on media. Also, the use of genetic engineering tools will enable those skilled in the art to manipulate the genes for the purpose of altering the polypeptide product in any fashion feasible. Using the method of the present invention, in combination with available tools for protein engineering (e.g., site-directed mutagenesis), those skilled will be able to prepare a bioactive polypeptide having any desired level of toxicity, whether non-toxic, or of diminished, equal or greater toxicity than the native form.

The method of the invention provides a further step of expressing the cDNA under conditions in which the polypeptide is recovered in an inactive form due to the failure to form one or more disulfide bridges. As described in greater detail below, this step involves the avoidance of posttranslational processes that would otherwise serve to form such linkages.

Optionally, and preferably, the method provides a further step of treating the inactivated bioactive polypeptides in order to retain the cysteine residues and prevent the spontaneous formation of disulfide bonds. A preferred treatment includes ozone treatment, in the manner described herein. Ozonation affects the cysteine residues by converting the pendent sulfhydryl (—SH) groups to corresponding —SO3X groups, which, unlike the sulfhydryl groups, are unable to form a disulfide bridge. Such treatment is not necessary, however, for those inactivate polypeptides that are found to not spontaneously reform, and that provide the desired activity. Ozonation is preferred for polypeptides such as neurotoxins, where Applicant has shown that upon cleavage and ozonation of the sulfhydryl groups, native neurotoxins are both stable and active.

The invention further includes a delivery formulation comprising a bioactive polypeptide that has been rendered inactive by virtue of the failure to form one or more disulfide bridges. Such polypeptides can be stably stored and used under conditions in which disulfide bonds are prevented from spontaneously reforming.

In yet another aspect, the invention provides a method of administering a bioactive polypeptide to a host, comprising the step of providing the polypeptide in an inactive form and within a suitable composition, and administering the composition to a host. In a related aspect, the invention provides a host having administered such a polypeptide. Compositions of the present invention can be used for a variety of purposes. Compositions are particularly useful in situations calling for a polypeptide in a form that is as close to native as possible, yet without an unwanted bioactivity.

In preparing a delivery formulation of this invention, in a preferred embodiment, the immunokine is formulated in physiological solution such as 0.9% sodium chloride (saline) or buffered saline (e.g., disodium hydrogen phosphate and citric acid) with a pH of between about 4.4 and about 6.5. The immunokine is added to achieve a final concentration effective for its intended use. Typically, for instance where the immunokine is in the form of deactivated alpha cobratoxin, the immunokine is added to a final concentration of between about 100 micrograms/ml (1.28 E-4M) to about 1000 micrograms/ml (1.28E-5M), and preferably between about 500 micrograms/ml and about 700 micrograms/ml.

Benzalkonium chloride (MW 360, and 375 as determined by perchloric acid titration) is described variously as a cationic surfactant (see, e.g., Drug Development Research 40:65-76 (1997) and a cationic disinfectant (J. Orthop. Trauma. 11:121-125 (1997), and is widely used as an antimicrobial agent in pharmaceuticals, particularly opthalmic preparations. The quaternary ammonium salt, e.g., benzalkonium chloride (CAS 8001-54-5) confers upon the immunokine the ability to pass through the mucous membrane comprising the hard and soft palate of the buccal cavity, and into circulating blood.

The delivery formulation is preferably provided within a delivery device, e.g., aerosol and non-aerosol (e.g., pump spray) dispensers as are commonly used for non-fragrance and OTC products. Delivery devices useful in the delivery system of this invention are available from a variety of sources. Representative, and preferred aerosol actuators are available, for instance, from Valois SA in the form of their line of “protruding actuators”. The delivery device preferably provides an optimal combination of such features as package design and functionality, stability and ease of use.

In a particularly preferred embodiment, the formulation is applied (e.g., sprayed on) the roof of the mouth in a volume of about 0.1 ml, and preferably between about 0.05 ml and about 0.15 ml.

While the invention has been explained in relation to its preferred embodiments, it is to be understood that various modifications thereof will become apparent to those skilled in the art. The foregoing disclosure is not intended or to be construed to limit the present invention, or to otherwise exclude any such other embodiments, adaptations, variations and equivalent embodiments.

1-69. (canceled)

70. A medicament delivery method comprising;
(a) providing a delivery system comprising a delivery formulation comprising an effective amount of an inactivated bioactive peptide and an effective amount of a mucosal absorption enhancer comprising a quaternary ammonium salt for enhancing mucosal absorption of the peptide in the buccal cavity;
(b) bringing the delivery formulation into contact with the mucosal surface of the buccal cavity under conditions suitable to permit an effective amount of the peptide to be absorbed, wherein the quaternary ammonium salt is used at a final concentration of about 0.005% and about 0.05%, based on the weight of the formulation.

71. The method of claim 70 wherein the quaternary ammonium salt comprises benzalkonium chloride.

72. The method of claim 70 wherein the peptide comprises an ozone-inactivated toxin.

73. The method of claim 70 wherein the formulation is delivered by spraying to the roof of the mouth.

74. The method of claim 70 wherein the delivery system further comprises an aerosol actuator, for use in containing and spraying the delivery formulation.

75. A delivery system comprising a dispenser containing a delivery formulation comprising an effective amount of an inactivated bioactive peptide and an effective amount of a mucosal absorption enhancer comprising a quaternary ammonium salt for enhancing mucosal absorption of the peptide in the buccal cavity, wherein the quaternary ammonium salt is used at a final concentration of about 0.005% and about 0.05%, based on the weight of the formulation.

76. The system of claim 75 wherein the dispenser is selected from the group consisting of aerosol and non-aerosol dispensers.

77. A medicament delivery formulation comprising an effective amount of an inactivated bioactive peptide and an effective amount of a mucosal absorption enhancer comprising a quaternary ammonium salt for enhancing mucosal absorption of the peptide in the buccal cavity, wherein the quaternary ammonium salt is used at a final concentration of about 0.005% and about 0.05%, based on the weight of the formulation.

78. A combination comprising a delivery formulation according to claim 77 in contact with the mucosal membrane of the roof of the mouth.

79. The method of claim 70 wherein the peptide has a molecular weight of at least 500 daltons.

80. The method of claim 70 wherein the quaternary ammonium salt comprises a tetrasubstituted ammonium salt, in which the substituent groups comprise hydrocarbon compounds attached to the nitrogen by N—C bonds.

81. The method of claim 70 wherein the bioactive peptide is inactivated by a method comprising the steps of treating the peptide with ozone under conditions suitable to oxidize any disulfide bonds in order to form corresponding pairs of cysteic acid residues, and then stabilizing the resultant cysteic acid residues and preventing the reformation of disulfide bonds.

82. The method of claim 81 wherein the inactivated bioactive peptide retains one or more properties selected from the group consisting of immunogenicity and anti-viral activity.

83. The method of claim 70 wherein the bioactive peptide is selected from the group consisting of toxins affecting the presynaptic neurojunction, toxins affecting the postsynaptic neurojunction, toxins affecting ion channels, and toxins that damage the cell membrane.

84. The method of claim 83 wherein the toxins affecting the presynaptic neurojunction toxins are selected from the group consisting of notexin, β-bungarotoxin, crotoxin, taipoxin, textilotoxin and α-latrotoxin.

85. The method of claim 83 wherein the toxins affecting the postsynaptic neurojunction are selected from the group consisting of α-conotoxins, α-cobrotoxin, erabutoxin, α-cobratoxin and α-bungarotoxin.

86. The method of claim 83 wherein the toxins affecting ion channels are selected from the group consisting of dendrotoxins, scorpion toxins, m-conotoxins, and sea anemone toxins.

87. The method of claim 83 wherein the toxins that damage the cell membrane are membrane-damaging toxins selected from the group consisting of myotoxins, cardiotoxins, mellitin, and phospholipases.

88. The method of claim 82 wherein the bioactive peptide is selected from the group consisting of protein hormones and enzymes.

89. The method of claim 88 wherein the bioactive peptide is a protein hormone selected from the group consisting of oxytocin, arginine vasopressin, insulin, growth hormone and calcitonin.
